(19) United States
(12) Patent Application Publication
(10) Pub. No.: US 2014/0127316 A1
(43) Pub. Date: May 8, 2014

(54) PROPOLIS AND CAFFEIC ACID PHENETHYL ESTER AND USES THEREOF

(71) Applicant: NEW YORK UNIVERSITY, New York, NY (US)

(72) Inventors: Coral Omene, Bronx, NY (US); Owen O'Connor, Scarsdale, NY (US); Krystyna Frenkel, New York, NY (US)

(21) Appl. No.: 14/118,327

(22) PCT Filed: May 21, 2012
(86) PCT No.: PCT/US12/38777

§ 371 Date: Jan. 22, 2014

Related U.S. Application Data
(60) Provisional application No. 61/519,325, filed on May 20, 2011.

(51) Int. Cl. A61K 35/64; A61K 45/06; A61K 31/235

(52) U.S. Cl. CPC A61K 35/644; A61K 31/235; A61K 45/06
USPC 424/539; 514/532

(57) ABSTRACT

Methods for treating a subject with cancer using a combined therapeutic regimen comprising administering propolis or caffeic acid phenethyl ester (CAPE) in conjunction with other cancer therapeutics are described herein. More particularly, methods for treating subjects with breast cancer using the combined therapeutic regimen are embodied herein. The present methods are particularly useful for treating cancer patients (e.g., breast cancer patients) who are refractory to or who have become refractory to the cancer therapeutic/s used in combination with propolis or CAPE. Propolis or CAPE for use in a combined treatment with other cancer therapeutics for treating cancer patients and compositions comprising propolis or CAPE and other cancer therapeutics are also encompassed herein wherein the ability of propolis or CAPE to act as a histone deacetylase (HDAC) inhibitor is used to advantage. Also encompassed herein are methods and compositions for the treatment of diseases caused by or associated with viral infections. In particular embodiments, methods and compositions for the treatment of viral infections caused by or associated with retroviruses are envisioned, wherein the ability of propolis or CAPE to act as a histone deacetylase (HDAC) inhibitor is also used to advantage. Also encompassed herein are methods and compositions for the treatment of diseases caused by or associated with viral infections.

### GOVERNMENT RIGHTS

This invention was made with government support under Grant No. ES00260 awarded by the National Institutes of Health. Accordingly, the United States Government has certain rights in the invention.

### FIELD OF THE INVENTION

The present invention relates to the field of cancer biology and agents for treatment of cancer. More particularly, the invention relates to a method for treating a subject with breast cancer using a combined therapeutic regimen comprising administering propolis or caffeic acid phenethyl ester (CAPE), based on the novel mechanistic finding described below showing the ability of propolis or CAPE to act as histone deacetylase (HDAC) inhibitors, in conjunction with agents used in hormonal therapy directed to the treatment of breast cancer. As described herein, such agents include, but are not limited to: agents that inhibit the activity of estrogen (e.g., Tamoxifen and Aromatase Inhibitors) and agents that inhibit the activity of epidermal growth factor receptor (EGFR). Also encompassed are compositions comprising propolis or CAPE in combination with, for example, agents that inhibit the activity of estrogen (e.g., tamoxifen) and agents that inhibit the activity of epidermal growth factor receptor (EGFR). Also encompassed herein are methods and compositions for the treatment of diseases caused by or associated with viral infections. In particular embodiments, methods and compositions for the treatment of viral infections caused by or associated with retroviruses are envisioned, wherein the ability of propolis or CAPE to act as histone deacetylase (HDAC) inhibitors is used to advantage. Human immunodeficiency virus type 1 (HIV-1) is an exemplary retrovirus treatable with methods and compositions described herein (Archin N M et al, 2009; Wightman F et al 2011).

### BACKGROUND OF THE INVENTION

Breast cancer (BC) mortality is second only to lung cancer among women. Response rates in patients with metastatic BC progressively decline with advancing lines of treatment. Of the patients eligible for first-line endocrine therapy, approximately 30% have an objective response to treatment, with median time to progression (TTP) of 9-11 months (Gibson L J et al 2007). Generally, 30-65% of patients respond to first-line anthracycline- or taxane-based chemotherapy, with a median TTP of 7-8 months (Conlin A K and Seidman A D 2007, Jones S E 2008). Yet, after failure of one line of chemotherapy, response rates decline with TTP of ˜4 months (Conlin A K and Seidman A D et al 2007, Jones S E 2008). While targeted anticancer therapies such as trastuzumab, bevacizumab, and lapatinib have improved metastatic BC outcomes, these agents are also subject to mechanisms involved in cancer resistance (Wong S T and Goodin S, 2009).

In light of the above, there is a need for alternative therapeutic regimens for breast cancer patients, particulary those who have developed resistance to first-line therapeutic approaches.

The citation of references herein shall not be construed as an admission that such is prior art to the present invention.

Several publications and patent documents are referenced in this application in order to more fully describe the state of the art to which this invention pertains. The disclosure of each of these publications and documents is incorporated by reference herein.

Other features and advantages of the invention will be apparent from the detailed description, the drawings, and the claims.

### SUMMARY OF THE INVENTION

The present inventors demonstrate herein that CAPE inhibits histone deacetylases (HDAC) in a manner similar to that shown for two FDA-approved agents (Vorinostat and Romidepsin) targeting lymphoma. This finding was also observed with propolis, making CAPE and propolis a naturally-occurring epigenetic therapy. HDAC inhibition enables breast cancer (BC) cells to become responsive to therapy and based on its safety record, propolis is predicted to have a favorable therapeutic effect in women with advanced, treatment-refractory BC. The present findings reveal that treatment with either propolis or CAPE: 1) decreases expression of hormone receptors in hormone receptor positive (HR+) cells; 2) turns on HR expression in hormone receptor negative (HR−) cells; and 3) decreases the expression of EGFR proteins in HR− cells and p-Her2 in Her2+ cells, which may all contribute to a favorable therapeutic outcome.

More particularly, the data presented herein show that CAPE and propolis induce decreases in hormone receptors, estrogen receptor (ER) and progesterone receptor (PR), suggesting that this decrease in expression could lead to anti-hormonal effects in hormonal therapy refractory cells of estrogen receptor positive (ER+)/progesterone receptor positive (PR+) patients. The re-expression of ER protein in triple-negative breast cancer (TNBC; ER−, PR−, Her2−) cells following treatment with propolis or CAPE could render these TNBC patients susceptible to anti-estrogen therapy in either the adjuvant or metastatic setting as well as for chemoprevention. Decreases in over-expression of EGFR in TNBC and phosphorylated Her2/neu in Her2-overexpressing BC observed following treatment with propolis or CAPE suggest that these are excellent candidates for targeted therapy in patients who have TNBC or progressed on anti-Her2 therapy like Herceptin or Lapatinib for Her2 positive patients.

Accordingly, a method for treating a patient with cancer is described herein, the method comprising administering to the patient a therapeutically effective amount of propolis or CAPE in combination with an agent used in hormonal therapy of cancer, wherein administration of the propolis or CAPE and the agent reduces the number of cancer cells or the tumor burden in the patient, thereby treating the patient. Also encompassed herein is a therapeutically effective amount of propolis or CAPE for use in a combined treatment with an agent used in hormonal therapy of cancer for treating a patient with cancer, wherein administration of the propolis or CAPE and the agent reduces the number of cancer cells or the tumor burden in the patient, thereby treating the patient. In particular embodiment of the method or use, the cancer is breast cancer or prostate cancer. In particular embodiment of the method or use, the breast cancer is a triple negative breast cancer (TNBC). In a more particular embodiment of the method or use, the agent is an anti-estrogen therapeutic. Exemplary anti-estrogen therapeutics include, without limitation, Tamoxifen, Faslodex and Aromatase Inhibitors letrozole (Femara), anastrazole (Arimidex) and exemestane (Aromasin). In keeping with results presented herein, the propolis or CAPE restores sensitivity to the anti-estrogen therapeutic by inducing re-expression of estrogen receptors on the TNBC.

In a further embodiment, the method or use relates to a triple negative breast cancer (TNBC) that over-expresses epidermal growth factor receptor (EGFR). In accordance with results presented herein, the propolis or CAPE restores sensitivity to the EGFR inhibitor in TNBC that is refractory to EGFR inhibition. In particular embodiment of the method or use, the agent is an EGFR inhibitor. Exemplary EGFR inhibitors include cetuximab (Erbitux), Panintumumab (Vectibix), Erlotinib (Tarceva).

In a further embodiment, the method or use relates to a cancer that is refractory to at least one therapeutic regimen directed to eradicating the cancer. In a particular embodiment thereof, the cancer is a breast cancer that is refractory to hormonal therapy. In a more particular embodiment, the breast cancer is Her2+. In an even more particular embodiment, the Her2+ breast cancer over-expresses Her2+. In a particular aspect of the method or use, the breast cancer is refractory to an anti-Her 2 signaling drug and the agent is the anti-Her 2 signaling drug and the propolis or CAPE restores sensitivity to anti-Her 2 signaling drugs. Exemplary anti-Her 2 signaling drugs include trastuzumab (Herceptin), Lapatinib and Pertuzumab.

In another aspect, the methods and uses described herein further comprise treating the patient with chemotherapy or use in a combined treatment that includes chemotherapy.

In yet another aspect, the method for treating a cancer patient or use in combined treatment of same comprises administering to the patient a therapeutically effective amount of propolis or CAPE and measuring HDAC activity in the patient or in a sample isolated from the patient, wherein said measuring HDAC activity reflects efficacy of the propolis or CAPE administered and wherein administration of propolis or CAPE and the agent reduces the number of cancer cells or the tumor burden in the patient.

Also encompassed herein is a composition that comprises propolis or CAPE and an agent used in hormonal therapy of cancer and a pharmaceutically acceptable excipient. Methods of using the compositions described herein and uses thereof in combined treatment regimens are also envisioned.

In another aspect, a method for treating a patient with cancer, wherein the cancer is refractory to at least one therapeutic regimen directed to eradicating the cancer is described, the method comprising administering to the patient a therapeutically effective amount of propolis or CAPE in combination with an agent used in the at least one therapeutic regimen directed to eradicating the cancer, wherein administration of the propolis or CAPE and the agent reduces the number of cancer cells or the tumor burden in the patient, thereby treating the patient. In certain embodiments, the cancer is prostate cancer, head and neck cancer, melanoma, lung cancer or leukemia. In more particular embodiments thereof, the cancer is prostate cancer and the prostate cancer is refractory to anti-androgens (Lupron, Zoladex, Casodex, nilandrone, flutamide, abiraterone); the cancer is head and neck cancer and the head and neck cancer is refractory to Cetuximab or chemotherapy+/−radiation; the cancer is melanoma and the melanoma is refractory to chemotherapy or Ipilumumab or BRAF inhibitors such as vemurafenib; the cancer is lung cancer and the lung cancer is refractory to chemotherapy or Alimta or Erlotinib (Tarceva); or the cancer is leukemia and the leukemia is refractory to chemotherapy.

In a further aspect, a method for treating a patient with cancer, wherein the cancer is refractory to at least one therapeutic regimen directed to eradicating the cancer is described, the method comprising administering to the patient a therapeutically effective amount of propolis or CAPE in combination with an agent used in the at least one therapeutic regimen directed to eradicating the cancer, wherein administration of the propolis or CAPE and the agent reduces the number of cancer cells or the tumor burden in the patient, thereby treating the patient. Also envisioned herein is a therapeutically effective amount of propolis or CAPE for use in a combined treatment with an agent used in a therapeutic regimen directed to eradicating the cancer for treating a patient with cancer, wherein the cancer is refractory to the therapeutic regimen directed to eradicating the cancer, the method comprising administering to the patient a therapeutically effective amount of propolis or CAPE in combination with an agent used in the therapeutic regimen directed to eradicating the cancer, wherein administration of the propolis or CAPE and the agent reduces the number of cancer cells or the tumor burden in the patient, thereby treating the patient.

In another aspect, a method for treating a patient with cancer, wherein the cancer is refractory to hormonal therapy is described, the method comprising administering to the patient a therapeutically effective amount of propolis or CAPE in combination with an agent used in the hormonal therapy of cancer, wherein administration of the propolis or CAPE and the agent reduces the number of cancer cells or the tumor burden in the patient, thereby treating the patient. Use of propolis or CAPE and an agent used in hormonal therapy of cancer in a combined treatment for treating a cancer patient with a cancer that is refractory to hormonal therapy is also encompassed herein.

In yet another aspect, a method for treating a patient with breast cancer, wherein the breast cancer is refractory to hormonal therapy is described, the method comprising administering to the patient a therapeutically effective amount of propolis or CAPE in combination with an agent used in the hormonal therapy of breast cancer, wherein the administering of the propolis or CAPE and the agent reduces the number of cancer cells or the tumor burden in the patient, thereby treating the patient. Use of propolis or CAPE and an agent used in hormonal therapy of breast cancer in a combined treatment for treating a cancer patient with a breast cancer that is refractory to hormonal therapy is also encompassed herein. In an embodiment thereof, the method or use calls for agents that are anti-estrogen therapeutics, such as, e.g., Tamoxifen, Faslodex and Aromatase Inhibitors. Under such circumstances, the propolis or CAPE restores sensitivity to the anti-estrogen therapeutic by inducing re-expression of estrogen receptors on the breast cancer. In an embodiment thereof, the method or use relates to a breast cancer that is Her2+. In a further embodiment thereof, the breast cancer over-expresses Her2+. In particular embodiments, the method or use pertains to circumstances wherein the breast cancer is refractory to an anti-Her 2 signaling drug and the agent used in combination with propolis or CAPE is the anti-Her 2 signaling drug. In accordance with results presented herein, the propolis or CAPE restores sensitivity to the anti-Her 2 signaling drug. Exemplary anti-Her 2 signaling drugs include trastuzumab (Herceptin) and Lapatinib.

Also encompassed herein is a method for treating a patient with Her2+ breast cancer, wherein the Her2+ breast cancer is refractory to an anti-Her2 signaling drug, the method comprising administering to the patient a therapeutically effective amount of propolis or CAPE in combination with the anti-Her2 signaling drug, wherein the combination of propolis or CAPE and the anti-Her2 signaling drug reduces the number of cancer cells or the tumor burden in the patient, thereby treating the patient. As results presented herein demonstrate, the propolis or CAPE restores sensitivity to the anti-Her2 signaling drug. As described herein, exemplary anti-Her 2 signaling drugs include trastuzumab (Herceptin) and Lapatinib.

Also encompassed herein is a method for treating a patient with triple negative breast cancer (TNBC) that over-expresses epidermal growth factor receptor (EGFR), the method comprising administering to the patient a therapeutically effective amount of propolis or CAPE in combination with an EGFR inhibitor, wherein administration of the propolis or CAPE and the EGFR inhibitor reduces the number of cancer cells or the tumor burden in the patient, thereby treating the patient. In a particular embodiment thereof, the breast cancer is refractory to EGFR inhibitors. Propolis or CAPE for use in a combined treatment with an EGFR inhibitor for treating a patient with a cancer that is refractory to EGFR inhibitors is also envisioned. In keeping with results presented herein, the method and use benefit from the properties of propolis and/or CAPE whereby sensitivity to the EGFR inhibitor is enhanced. Exemplary EGFR inhibitors include cetuximab (Erbitux), panitumumab (Vectibix) and Erlotinib (Tarceva).

In a further aspect, a method for treating a patient with triple negative breast cancer (TNBC), wherein the breast cancer is refractory to anti-estrogen therapeutics is described, the method comprising administering to the patient a therapeutically effective amount of propolis or CAPE in combination with an anti-estrogen therapeutic, wherein administration of the propolis or CAPE and the anti-estrogen therapeutic reduces the number of cancer cells or the tumor burden in the patient, thereby treating the patient. Propolis or CAPE for use in a combined treatment with an anti-estrogen therapeutic for treating a patient with a cancer that is refractory to anti-estrogen therapeutics is also envisioned. In accordance with findings presented herein, the propolis or CAPE restores sensitivity to the anti-estrogen therapeutic by inducing re-expression of estrogen receptors on the TNBC cells. Exemplary anti-estrogen therapeutics include, without limitation, Tamoxifen, Faslodex and the drugs belonging to the class of aromatase inhibitors.

Methods and uses described herein may further comprise treating the patient with chemotherapy or use in a combined treatment that includes chemotherapy.

Also encompassed herein are methods, uses, and compositions for the treatment of diseases caused by or associated with viral infections. The methods and compositions for the treatment of viral infections caused by or associated with viral infections are envisioned based on the ability of propolis and CAPE to possess antiviral properties likely through their ability to act as histone deacetylase (HDAC) inhibitors. In particular embodiments, methods and compositions for the treatment of viral infections caused by or associated with retroviruses are envisioned, wherein the ability of propolis or CAPE to act as HDAC inhibitors is used to advantage. Human immunodeficiency virus type 1 (HIV-1) is an exemplary retrovirus treatable with methods and compositions described herein. With respect to the treatment of viral infections, which respond efficaciously to HDAC inhibitors, the methods and compositions of the invention comprise therapeutically effective amounts of propolis or CAPE either alone or in combination with anti-viral therapeutic agents. Viral infections, such as HIV-1, that undergo periods of latency wherein proviral quiescence is, for example, maintained, are exemplary targets for treatment with propolis or CAPE in accordance with results presented herein.

### BRIEF DESCRIPTION OF THE DRAWINGS

FIG. 1 illustrates cytotoxicity of (A) CAPE and (B) Propolis in breast cancer cell lines.

FIG. 2 illustrates the HDAC inhibitor activity of CAPE and Propolis by the hyperacetylation of histone proteins in the ER+/PR+ and TNBC cell lines (A), as well as in human peripheral blood mononuclear cells from a healthy volunteer after oral ingestion of CAPE-containing Propolis (B).

FIG. 3 shows the effects of CAPE and Propolis on breast cancer therapeutic targets in the ER+/PR+ cell line. The results of immunoblotting analyses of the indicated therapeutic targets (ER and PR) are depicted.

FIG. 4 shows the effects of CAPE and Propolis on breast cancer therapeutic targets. More particularly, CAPE exposure epigenetically results in the re-expression of a previously silenced ER gene and protein in MDA-231 TNBC cells comparable to the established histone deacetylase inhibitor (HDACi) LBH 589 (Panobinostat). In (A), ER a re-expression in MDA-231 (TNBC) cells is visualized by (top panels) immunofluorescence (receptor protein) and (bottom panel) RT-PCR (gene). In (B), immunoblotting analyses reveal that CAPE and propolis cause a reduction in EGFR expression in MDA-231 (TNBC) cells.

FIG. 5 shows the effects of CAPE and Propolis on breast cancer therapeutic targets. More particularly, immunoblotting analyses demonstrate that CAPE and Propolis cause a reduction in phosphorylated-Her2+ expression in SKBR3 (Her2+) cells.

FIG. 6 depicts the cytotoxicity of CAPE and Propolis in the Ly 1 lymphoma cell line.

FIG. 7 depicts the cytotoxicity of CAPE and Propolis for the RIVA lymphoma cell line.

FIG. 8 depicts the cytotoxicity of CAPE and Propolis for the Su-DHL6 lymphoma cell line.

FIG. 9 illustrates by Western Blotting CAPE induced hyperacetylation of Histone Proteins (Ac H3) in the indicated lymphoma cell lines (RIVA, LY 1, SU-DHL6).

### DETAILED DESCRIPTION OF THE INVENTION

Before the present discovery and methods of use thereof are described, it is to be understood that this invention is not limited to particular assay methods, or test compounds, and experimental conditions described, as such methods and compounds may vary. It is also to be understood that the terminology used herein is for the purpose of describing particular embodiments only, and is not intended to be limiting, since the scope of the present invention will be limited only by the appended claims.

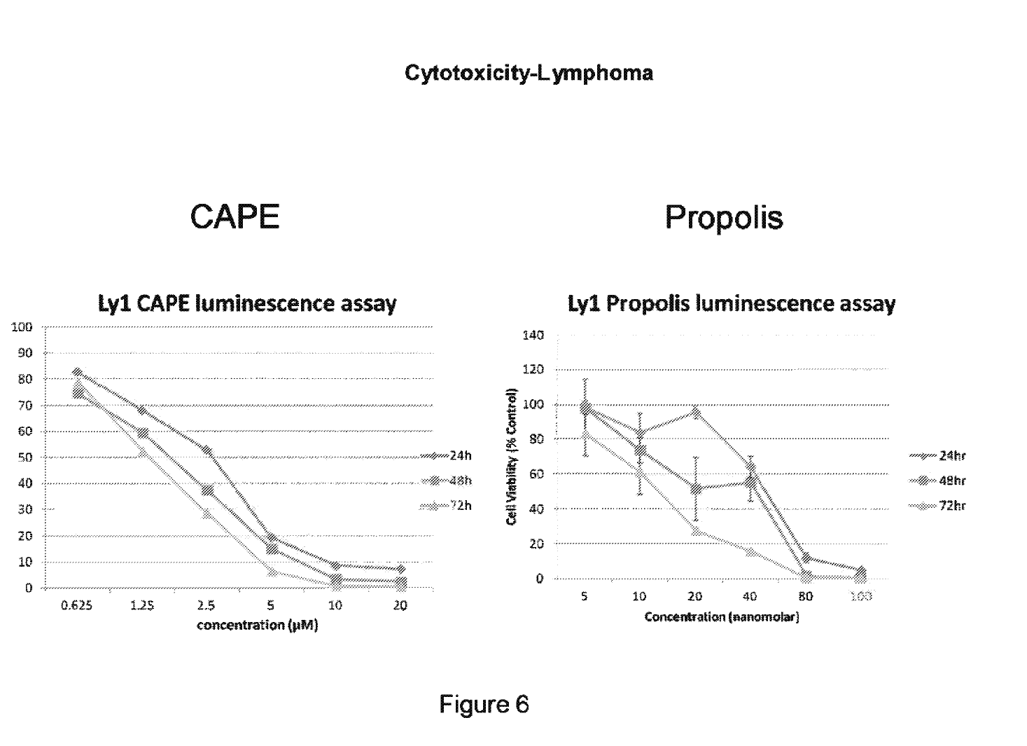

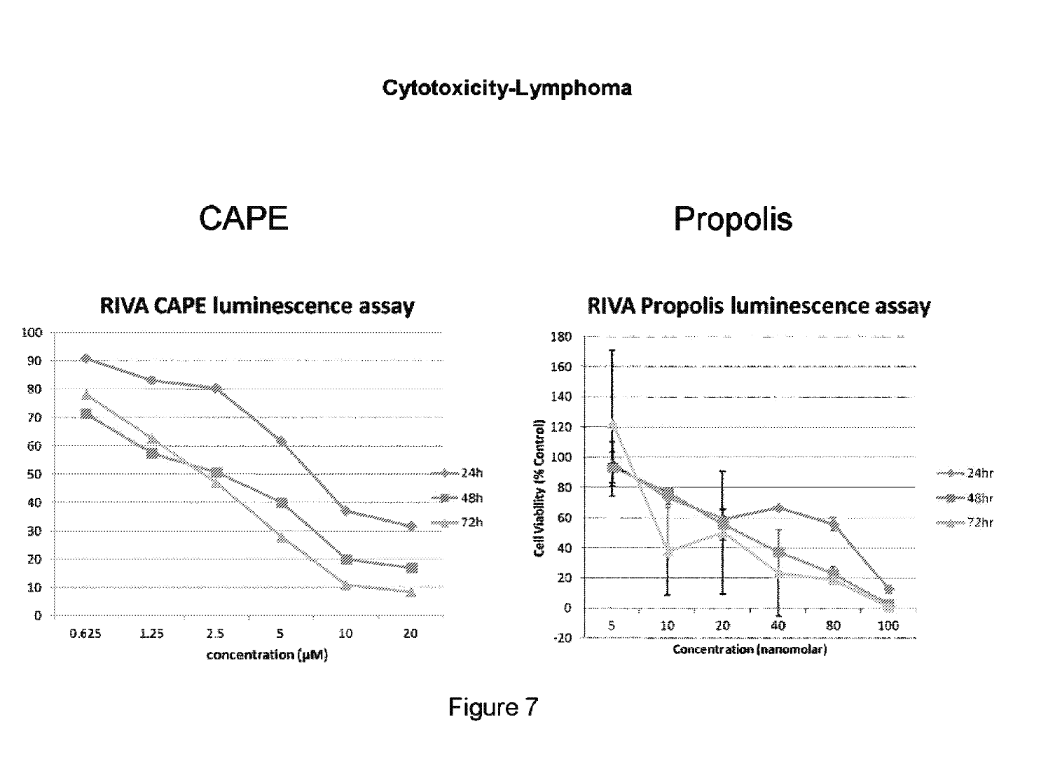

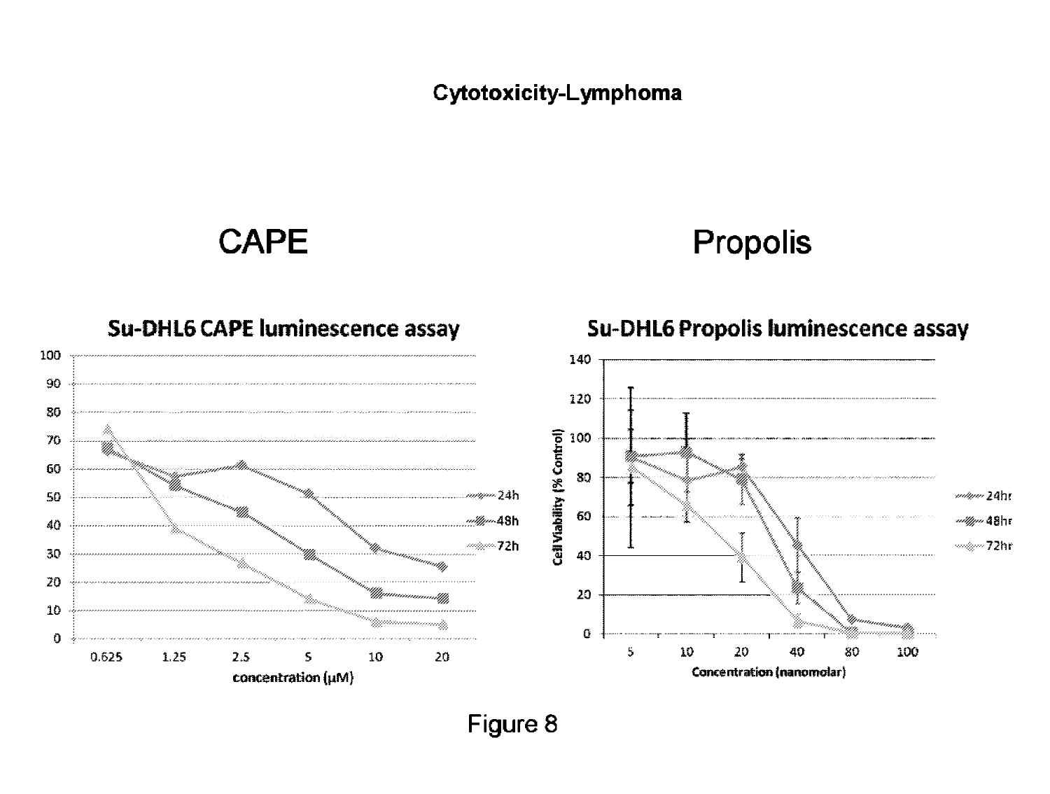

Propolis is an example of a naturopathic formulation derived from honeybees, which has been used safely for millennia around the world. One of the principal medicinal ingredients of propolis is caffeic acid phenethyl ester (CAPE). CAPE was found to be the major anti-cancer component in propolis (Grunberger D and Frenkel K, 2001) and has been associated with a variety of biological properties, including antibacterial, antiviral, antioxidant, anti-inflammatory, immunomodulatory, and anticancer effects. It is to be noted that there are different types of propolis and not all of them contain CAPE, for instance the Brazilian Propolis does not contain CAPE. Several studies have shown that CAPE exhibits marked in vitro and in vivo pre-clinical activity in a number of cancer models including the present inventors' data in breast cancer (Omene C et al 2011; Wu J and Omene C et al, 2011) and lymphoma (FIGS. 6-8, O'Connor O A et al, manuscript in preparation). Our laboratory was also among the first to describe the inhibitory effects of topically applied CAPE on DMBA-initiated and TPA-induced tumor promotion in mouse skin (Huang M T et al 1996). CAPE is thought to mediate its anticancer effects through a variety of mechanisms, though, notably, it is innocuous to normal cells.

The structure of CAPE is presented below:

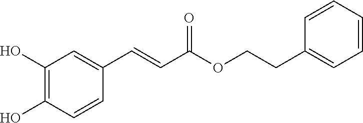 [SMILES: O=C(/C=C/c1ccc(O)c(O)c1)OCCc1ccccc1]

Molecular formula: C₁₇H₁₆O₄
Molecular weight: 284.3

As described herein, we have made the surprising discovery that the mechanism of the North American propolis and its main active ingredient, CAPE, is mediated by its ability to inhibit histone deacetylases (HDAC), thus making CAPE and propolis a naturally-occurring epigenetic therapy (Omene C et al, manuscript in preparation). We have previously shown that growth inhibition is seen not only in tumors induced by MCF-7 (ER+) BC cells, but also in MDA-MB-231 (MDA-231), triple-negative (ER−, PR−, Her2−) BC cells (TNBC), an aggressive phenotype known for its resistance to conventional cytotoxic drugs. In addition, a low dose of dietary CAPE inhibits growth of MDA-231 xenografts in nude mice, which appears to be even further suppressed by subsequent or concurrent topical CAPE treatment (Wu J and Omene C et al 2011). This growth inhibition has been attributed in part to both cell cycle arrest and the induction of frank apoptosis (Wu J and Omene C et al 2011). Since there are far fewer effective therapies for ER negative tumors, this finding becomes all the more important. Our gene expression microarray analysis has demonstrated that CAPE treatment down-regulates anti-apoptotic and proliferation genes, oncogenes, inflammatory cytokines, cyclins, growth factors, and multidrug resistance 1 (mdr-1) gene among others. Notably, we also have shown that CAPE inhibits NF-κB, an important transcription factor in cancer tumorigenesis, an effect that was patented in 1999 (Aggarwal B and Grunberger D, 1999). While the effects of CAPE alone are pleiotropic, our findings suggest that CAPE could also be useful as an adjuvant to conventional therapies such as Taxol, because low doses of CAPE alone, or in conjunction with Taxol serve as inhibitors of TNBC growth (Omene C et al, manuscript in preparation). We have also shown that CAPE decreases VEGF secretion by MDA-231 cells and inhibits tubule formation by endothelial cells, suggesting that it may also play a role in suppressing angiogenesis in vivo (Wu J and Omene C et al 2011). MDA-231 cell populations are thought to contain breast cancer stem cells (bCSCs), which are thought to be responsible for BC development, chemoresistance, recurrence and and metastasis. Our data show that CAPE limits the tumorigenic and growth potential of bCSCs derived from MDA-231 cells (Omene C et al, 2011).

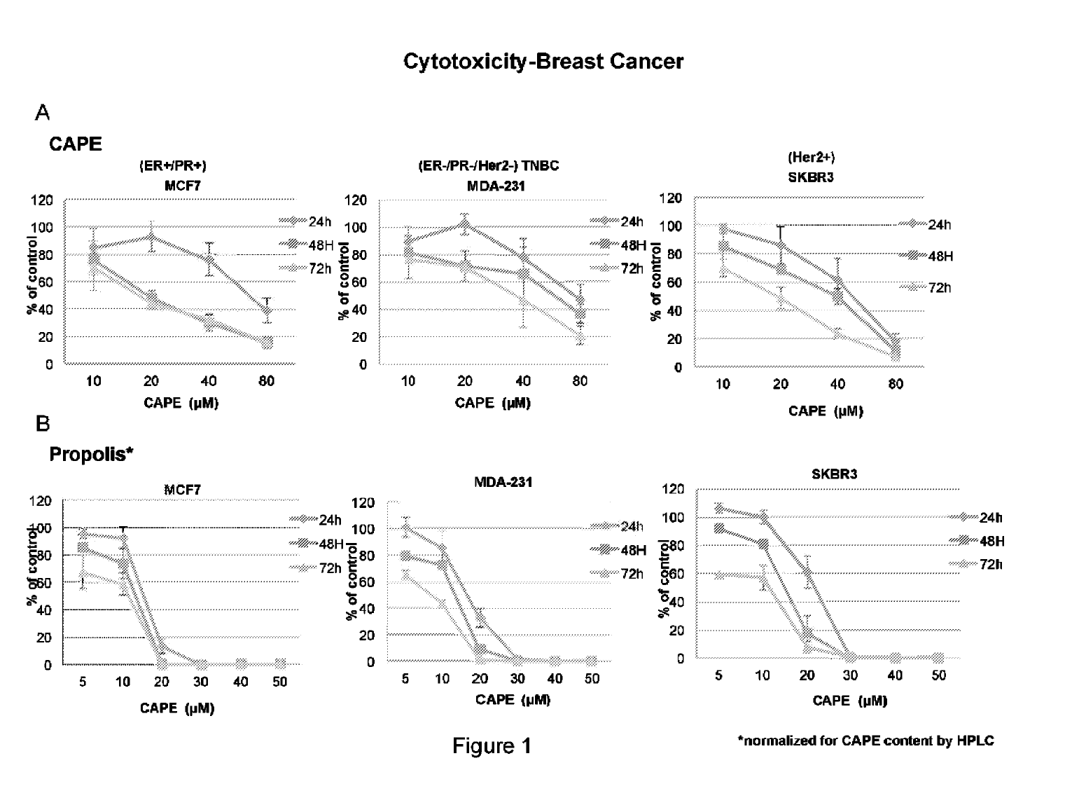

The current discovery that CAPE and propolis inhibit histone deacetylases is seen in several types of BC cell lines and causes a decrease in EGFR over-expression, as well as a re-expression of a silenced gene, ER, in MDA-231 cells (TNBC cells), as shown by immunofluorescence, decrease in ER and PR protein expression in MCF-7 (ER+/PR+) cells, and decrease in p-Her2 protein expression in SKBR3 (Her2+) cells. See FIGS. 1-5.

Inhibitors of HDAC enzymes alter patterns of gene expression, induce cellular differentiation, and promote cell cycle arrest and apoptosis (Marks P A et al, 2000). Many inhibitors of apoptosis are repressed by HDAC inhibitors and pro-apoptotic genes are activated (Mitsiades C S et al, 2004). Pharmacologic inhibition of histone deacetylation may therefore regulate gene expression patterns and, subsequently, cellular characteristics, making them attractive anticancer therapies. Currently, many patients and their caregivers are looking for alternative naturopathic therapies that may be associated with fewer adverse events with some potential to control drug- and hormone-resistant BC. Propolis, derived from honeybees, is a widely available natural substance with an extended safety record, having been available over-the-counter for many years, and is thus, an example of a naturopathic formulation that is safe, non-toxic, readily available, and affordable. Propolis represents an unusual formulation of a natural product that possesses properties of an epigenetic agent, which has the broader appeal of being viewed as ‘natural’, and possessing state-of-the-art therapeutic properties seen in many novel and innovative therapeutics being developed by the pharmaceutical industry.

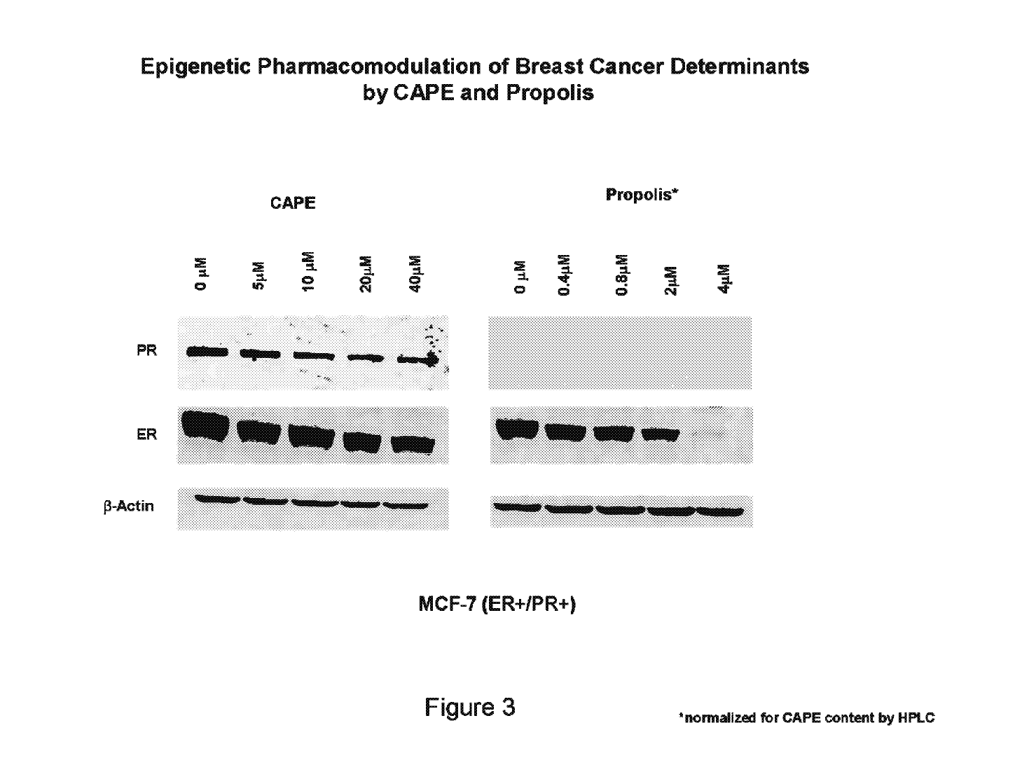

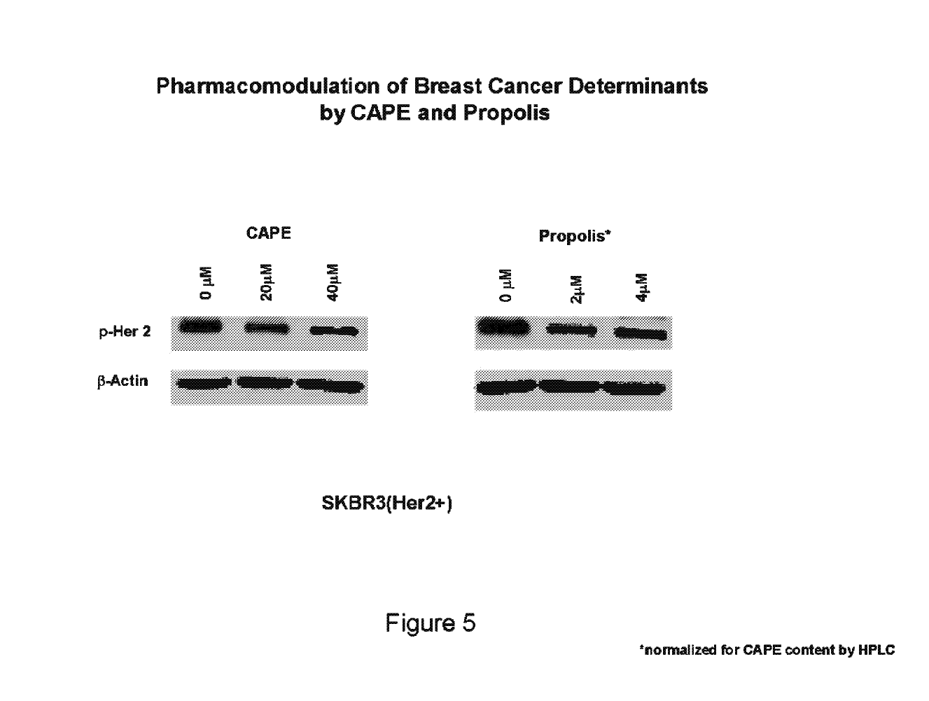

The HDAC inhibition seen in many BC cell lines by CAPE and propolis results in the decrease of EGFR over-expression and induction of ER in MDA-231 cells and decrease in ER and PR expression in MCF-7 cells (FIG. 3, 4). CAPE and Propolis cause a decrease in p-Her2, the activated form of Her 2 in SKBR3 cells, likely through intracellular signaling mechanisms (FIG. 5). These findings are reminiscent of the pleiotropic effects of HDAC inhibitors like vorinostat, aliphatic acids and depsipeptide in models of both epithelial and hematopoietic origin. To establish that the epigenetic effects of CAPE can be recapitulated in the natural product, we determined the concentration of CAPE in propolis by HPLC. These data show that when normalized for the concentration of CAPE in propolis, the HDAC inhibitory effects of the natural product, propolis, are in fact markedly superior to that seen with single agent CAPE. Interestingly, the aliphatic backbone structure of CAPE is within the expected structural class of chemicals known to possess this unusual activity. Furthermore, the enhanced effects of HDAC inhibition with propolis, compared to CAPE alone, make it a suitable candidate for a clinical trial in any area of oncology.

The research described herein advances our knowledge and presents a scientific platform for a natural product, propolis, in the era of drug development, with the obvious attractive qualities that include, extended safety history, wide availability, and affordability. All of these attributes lead to minimal added toxicity, especially in heavily pre-treated metastatic patients who already bear cumulative toxicities from prior treatment regimens in the setting of therapeutic benefit with the overall outcome being one of prolonged survival, decreased mortality, and increased quality of life.

Tamoxifen, a selective estrogen receptor modulator, is thought to work by competitive blockade of the estrogen receptor, thus inhibiting estrogen-dependent gene expression. Data suggest that there is significant cross-talk between the estrogen receptor and EGFR and HER2 signaling pathways, such that either de novo or acquired resistance to tamoxifen is driven by increased growth factor receptor signaling in the face of continued repression of estrogen receptor genomic action (Massarweh S et al, 2008). There is a great need for new agents that would increase survival of advanced BC patients and decrease treatment toxicity. All of the effects of CAPE/Propolis make it very attractive for use in heavily pre-treated metastatic BC patients who may likely benefit from therapy with minimal added toxicity, if any. CAPE-containing propolis (CP) fulfills this need based on its well documented safety and its role as an HDAC inhibitor. The subsequent decrease of hormone receptors in hormone-positive BC following propolis or CAPE treatment may potentially lead to anti-hormonal effects in hormone refractory cells or the re-expression of a previously silenced ER gene in TNBC may render these cells susceptible to anti-estrogen therapy which could be very important for chemo prevention in the adjuvant setting. Finally, the decrease in over-expression of EGFR in TNBC and phosphorylated Her 2/neu in Her2-overexpressing cells by CAPE-containing Propolis make it an excellent candidate as a targeted therapy in patients who have TNBC or have progressed on Herceptin for Her2 positive patients.

Results presented herein thus offer alternative therapeutic regimens for breast cancer patients. Indeed, this research provides a unique opportunity to explore the issue of treatment for refractory breast cancer patients using a novel approach with the use of a naturopathic formulation of CAPE containing propolis or CAPE, whose properties include down regulating the mdr-1 gene important for the development of drug resistance.

In order to more clearly set forth the parameters of the present invention, the following definitions are used:

As used in this specification and the appended claims, the singular forms “a”, “an”, and “the” include plural references unless the context clearly dictates otherwise. Thus, for example, reference to “the method” includes one or more methods, and/or steps of the type described herein and/or which will become apparent to those persons skilled in the art upon reading this disclosure and so forth.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity of the isolated polypeptide, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g., chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose-binding domain, calmodulin-binding protein, maltose-binding protein, chitin-binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

A “cell line” is a clone of a primary cell or cell population that is capable of stable growth in vitro for many generations.

The compositions containing the molecules or compounds of the invention can be administered for pharmaceutical or therapeutic purposes. In pharmaceutical or therapeutic applications, compositions are administered to a patient suffering from cancer (such as, e.g., breast cancer) in an amount sufficient to cure or at least partially arrest the symptoms of the disease and its complications. An amount adequate to accomplish this is defined as a “therapeutically effective amount or dose.” Amounts effective for this use will depend on the severity of the disease and the weight and general state of the patient.

In a further aspect of pharmaceutical or therapeutic applications, compositions are administered to a patient suffering from a viral infection (such as, e.g., an infection with HIV-1) in an amount sufficient to cure or at least partially arrest the symptoms of the disease and its complications. With respect to an infectious disease caused by a viral infection, the compositions and methods of the invention can be used to advantage to enhance immune responses to the viral infection or reduce viral load or the like. It is to be understood that the method and compositions of the invention can be used to enhance immune responses in a patient infected with a single type of virus or a plurality of types of different viruses.

An “immune response” signifies any reaction produced by an antigen, such as a protein antigen, in a host having a functioning immune system. Immune responses may be either humoral, involving production of immunoglobulins or antibodies, or cellular, involving various types of B and T lymphocytes, dendritic cells, macrophages, antigen-presenting cells and the like, or both. Immune responses may also involve the production or elaboration of various effector molecules such as cytokines, lymphokines, chemokines, and the like. Immune responses may be measured both in in vitro and in various cellular or animal systems.

As used herein, the ability to “enhance immune responses” refers to the ability of a molecule (e.g., CAPE or a derivative thereof) to promote or augment an immune response.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically-active portion of an immunloglobulin molecule such as those portions known in the art as Fab, Fab′, F(ab′)2 and F(v).

As used herein, the term “refractory to a therapeutic agent or regimen” refers to a condition wherein the disease or condition is or has become unresponsive or resistant to the therapeutic agent or regimen. With respect to cancer, e.g., the cancer cells may have ceased to respond favorably (e.g., cease to proliferate or die) to an anti-cancer therapeutic to which they previously responded and thus, are refractory to the anti-cancer therapeutic. Alternatively, a cancer may never have responded favorably to a particular anti-cancer therapeutic and thus, is also viewed as refractory to the anti-cancer therapeutic.

As used herein, the term “adjuvant therapy” refers to a cancer treatment (e.g., chemotherapy, radiation, or biological therapy) that is administered to reduce or eliminate potential residual disease that cannot be seen with the eye or in scans or felt. Adjuvant therapy may be utilized, for example, after a surgical procedure that has removed all visualizable cancer cells and such is administered to improve the outcome of patients, particularly those at high risk for relapse. In the case of breast cancer, for example, tamoxifen and aromatase inhibitors may be administered to a patient for 5-10 years in an adjuvant setting. The term “adjuvant setting” refers to a condition wherein no detectable disease is apparent in a patient following an initial treatment regimen.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

Therapeutic Uses of Propolis and/or CAPE in Combination Therapy

The invention provides for treatment of a cancer in a patient by administration of a therapeutically effective amount of propolis and/or CAPE in combination with agents used in hormonal therapy directed to the treatment of the particular cancer. In a particular embodiment, the cancer is breast cancer. As described herein, such agents include, but are not limited to: agents that inhibit the activity of estrogen (e.g., Tamoxifen, Faslodex and Aromatase inhibitors) and agents that inhibit the activity of epidermal growth factor receptor (EGFR) and Her 2. The components of the combination therapy may be administered concurrently or in a temporally discrete manner. Accordingly, propolis or CAPE could be administered at the same time or before or after the agent used in hormonal therapy of the cancer. The timing of administration may be determined, in part, based on the route of administration for the above components/compounds. Practitioners skilled in the art of administration of such therapeutic regimes (e.g., oncologists) will evaluate suitable administration protocols based on their experience.

The invention provides methods for treating patients afflicted with cancer comprising administering to a subject an effective amount of a compound or combinations of compounds as described herein. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Various delivery systems are known and can be used to administer a compound of the invention, e.g., encapsulation in liposomes, micro- or nano-particles, microcapsules, recombinant cells capable of expressing the compound, receptor-mediated endocytosis (see, e.g., Wu and Wu (1987) J. Biol. Chem. 262:4429-4432). Methods of introduction can be enteral or parenteral and include but are not limited to intradermal, intramuscular, intraperitoneal, intravenous, subcutaneous, intranasal, epidural, topical, and oral routes. The compounds can be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into a localized site of a bacterial infection, such as, for example, a boil or abcess.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., a target tissue or tumor, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

Methods for administering therapeutically effective amounts of CAPE and analogs thereof are described in U.S. Pat. No. 6,689,811 and US Patent Publication Number 2002/0188021, the contents of each of which are incorporated herein by reference in their entireties.

A variety of publications address the pharmacologic activity of CAPE and derivatives therefrom, as well as structure/function analyses, including details pertaining to CAPE's lipophilic properties, accordingly such information is known in the art. See, for example, Watanabe et al. (2011) J Pharm Pharmacol 63:1378-86; Touaibia et al. (2011) Mini Rev Med Chem 11:695-713; Mirzoeva et al. (1995) Biorg Khrim 21:143-51; Ramanauskiene et al. (2011) Medicina (Kaunas) 47:354-9; and Serafim et al. (2011) Chem Res Toxicol 24:763-74, the entire content of each of which is incorporated herein by reference in its entirety. Exemplary caffeic acid derivatives include: hexyl caffeate, caffeoylhexylamide, ferulic acid, hexyl ferulate, and feruloylhexylamide and are described in Serafim et al. (2011) Chem Res Toxicol 24:763-74.

### Pharmaceutical Compositions

The present invention also provides pharmaceutical compositions. Accordingly, compositions comprising propolis or CAPE in combination with, for example, agents that inhibit the activity of estrogen (e.g., Tamoxifen, Faslodex and Aromatase Inhibitors) and agents that inhibit the activity of epidermal growth factor receptor (EGFR) and Her 2. Such compositions comprise a therapeutically effective amount of an agent or agents, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin, incorporated in its entirety by reference herein. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups, such as those interacting with hydrochloric, phosphoric, acetic, oxalic, tartaric acids, etc., and those formed with free carboxyl groups, such as those derived from sodium, potassium, ammonium, calcium, ferric hydroxides, isopropylamine, triethylamine, 2-ethylamino ethanol, histidine, procaine, etc.

The amount of the compound of the invention, which will be effective in the treatment of a cancer can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 0-10, 10-20, 20-30, 30-40, 40-50, 50-100, 100-200, 200-300, or 400-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems systems as well as early phase clinical trials.

The average daily homeopathic propolis regimen is ˜1000 mg. However, there is extensive data to support the safety of in vivo ingestion of propolis even at significantly higher doses. Animal toxicology studies using propolis revealed no observed differences in body weight, skin changes, mortality, or internal organs at autopsy between treated and control mice. Propolis has a low order of acute oral toxicity with reported LD₅₀ ranging from 2000 to 7300 mg/kg in mice (Burdock G A, 1998; DeCastro S L and Higashi K O, 1995), the entire contents of which are incorporated herein by reference. Propolis represents an unusual formulation of a natural product that possesses the properties of an epigenetic agent, which has the broader appeal of being viewed as a ‘natural product’ possessing unique therapeutic properties seen in many of the most promising novel therapeutics being developed by the pharmaceutical industry.

Therapeutically effective amounts of CAPE and analogs thereof are also described in U.S. Pat. No. 6,689,811 and US Application Publication Number 2002/0188021, the entire contents of each of which are incorporated herein by reference in their entireties.

Suppositories generally contain active ingredient in the range of 0.5% to 10% by weight; oral formulations may contain 10% to 95% active ingredient, the exact range in patients will be better determined from clinical trials.

### Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### REFERENCES

Castaldo, S and Capasso, F. Propolis, an old remedy used in modern medicine. Fitoterapia, 73 Suppl 1: S1-6, 2002.
Ansorge S, Reinhold D and Lendeckel U. Propolis and some of its constituents down-regulate DNA synthesis and inflammatory cytokine production but induce TGF-beta 1 production of human immune cells, Z Naturforsch [C] 58:580-9, 2003.
Grunberger D and Frenkel K, Inhibition of cataracts and other disorders, U.S. Pat. No. 6,313,165, Nov. 6, 2001.
Huang, M T, Ma, W, Yen, P et al Inhibitory effects of caffeic acid phenethyl ester (CAPE) on 12-O-tetradecanoylphorbol-13-acetate-induced tumor promotion in mouse skin and the synthesis of DNA, RNA and protein in HeLa cells. Carcinogenesis, 17: 761-5, 1996.
Wu J*, Omene C*, Karkoszka J, Bosland M, Eckard J, Klein C B, Frenkel K. Caffeic acid phenethyl ester (CAPE), derived from a honeybee product propolis, exhibits a diversity of anti-tumor effects in pre-clinical models of human breast cancer. Cancer Lett. 2011 Sep. 1; 308(1):43-53. Epub 2011 May 13 (* share 1^{st} authorship equally).
Omene C O, Wu J, Frenkel K. Caffeic Acid Phenethyl Ester (CAPE) derived from propolis, a honeybee product, inhibits growth of breast cancer stem cells. Invest New Drugs. 2011 May 3. [Epub ahead of print]
Aggarwal B. and Grunberger D. Inhibition of Nuclear Transcription Factor NF-kB by caffeic acid phenethyl ester (CAPE), Derivatives of CAPE, Capsaicin (8-methyl-nvanillyl-6-nonenamide) and resiriferatoxin, U.S. Pat. No. 5,981,583, Nov. 9, 1999.
Marks P A, Richon V M, Rifkind R A. Histone deacetylase inhibitors: inducers of differentiation or apoptosis of transformed cells. J Natl Cancer Inst; 92: 1210-6, 2000.
Mitsiades C S, Mitsiades N S, McMullan C J, et al. Transcriptional signature of histone deacetylase inhibition in multiple myeloma: biological and clinical implications. Proc Natl Acad Sci USA 2004; 101: 540-5.
Omene C et al., Propolis and its active component, Caffeic Acid Phenethyl Ester (CAPE), Epigenetically Pharmacomodulate Breast Cancer Therapeutic Targets (Manuscript in preparation).
Gibson L J, Dawson C K, Lawrence D H, Bliss J M. Aromatase inhibitors for treatment of advanced breast cancer in postmenopausal women. Cochrane Database Syst Rev; 1:CD003370, 2007.
Conlin A K, Seidman A D. Taxanes in breast cancer: an update. Curr Oncol Rep; 9:22-30, 2007.
Jones S E. Metastatic breast cancer: the treatment challenge. Clin Breast Cancer 2008; 8:224-33.
Wong S T and Goodin S. Overcoming drug resistance in patients with metastatic breast cancer. Pharmacotherapy. 2009 August; 29(8):954-65.
Massarweh S, Osborne C K, Creighton C J, et al. Tamoxifen resistance in breast tumors is driven by growth factor receptor signaling with repression of classic estrogen receptor genomic function. Cancer Res 2008; 68:826-33.
Burdock G A, Review of the biological properties and toxicity of bee propolis (propolis). Food chem toxicology 1998; April; 36(4):347-63.
DeCastro S L and Higashi K O, Effect of different formulations of propolis on mice infected with Trypanosoma cruzi. J Ethnopharmacol. 1995 April; 46(1):55-8).
Archin N M, Espeseth A, Parker D, Cheema M, Hazuda D, Margolis D M. Expression of latent HIV induced by the potent HDAC inhibitor suberoylanilide hydroxamic acid. AIDS Res Hum Retroviruses. 2009 February; 25(2):207-12.
Fiona Wightman, Paula Ellenberg, Melissa Churchill and Sharon R Lewin, HDAC inhibitors in HIV Immunology and Cell Biology (2012) 90, 47-54; doi:10.1038/icb.2011.95; published online 15 Nov. 2011.

The following protocols are provided to facilitate the practice of the present invention.

Examples described herein are put forth so as to provide those of ordinary skill in the art with a complete disclosure and description of how to make and use the assay, screening, and therapeutic methods of the invention, and are not intended to limit the scope of what the inventors regard as their invention. Efforts have been made to ensure accuracy with respect to numbers used (e.g., amounts, temperature, etc.), but some experimental errors and deviations should be accounted for. Unless indicated otherwise, parts are parts by weight, molecular weight is average molecular weight, temperature is in degrees Centigrade, and pressure is at or near atmospheric.

### Example I

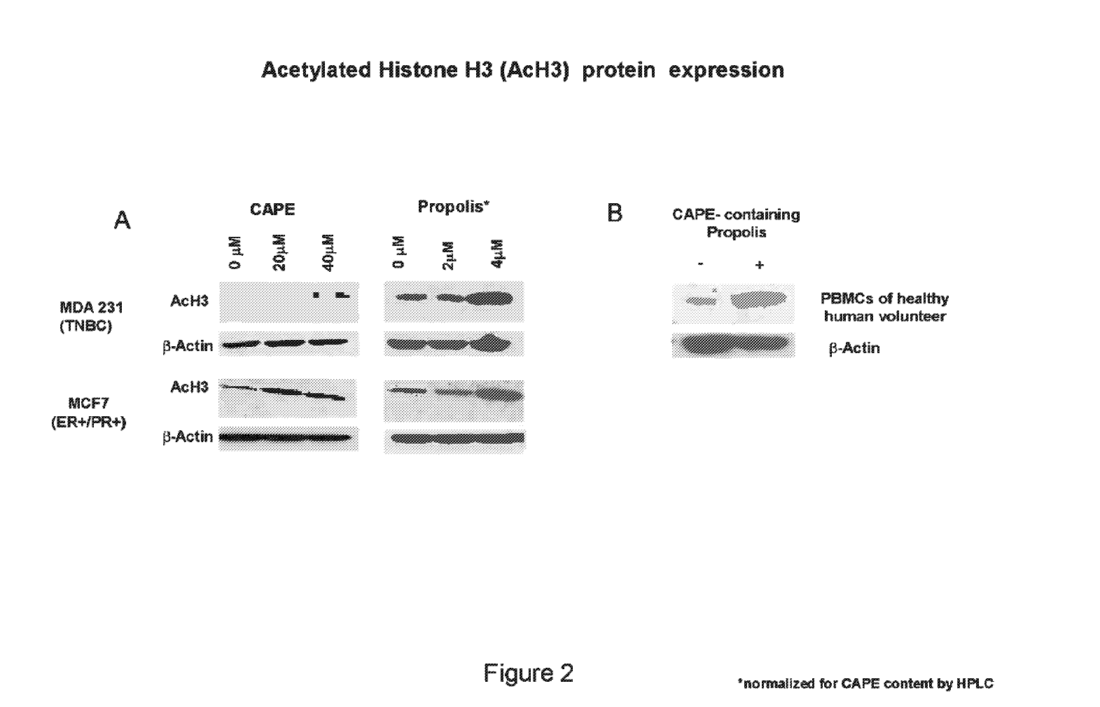

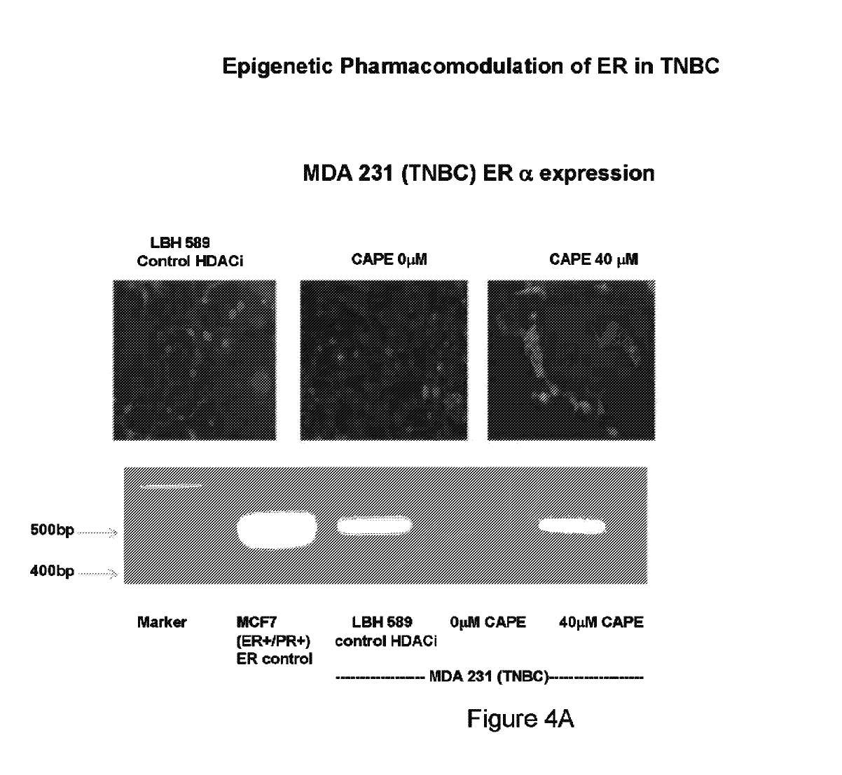

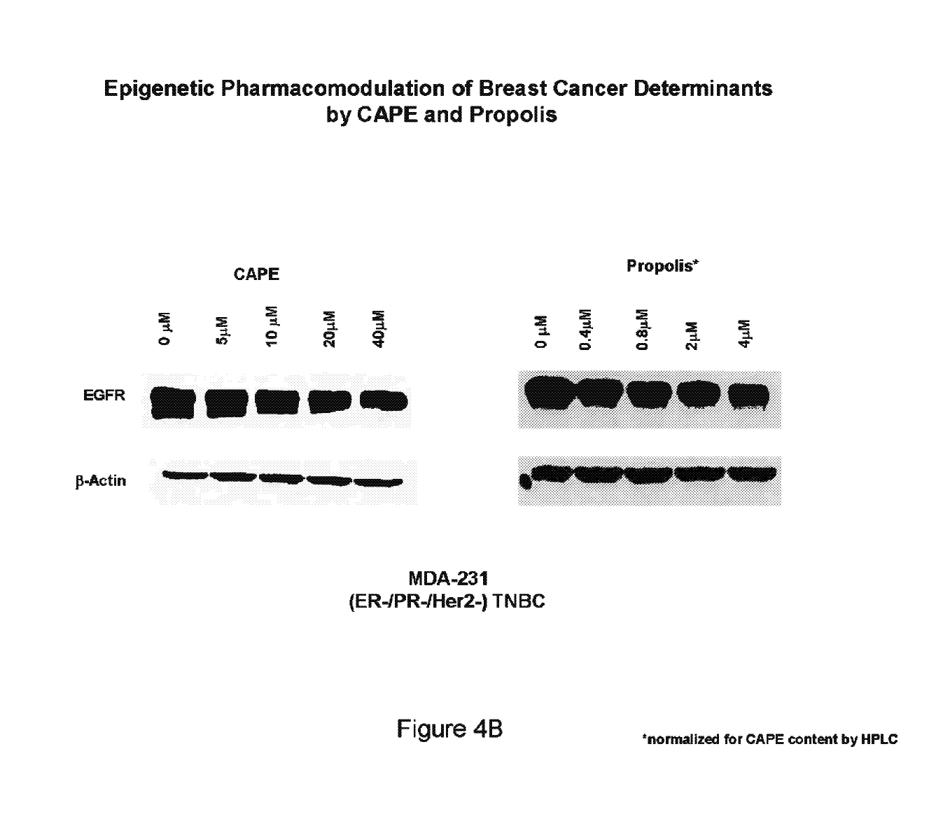

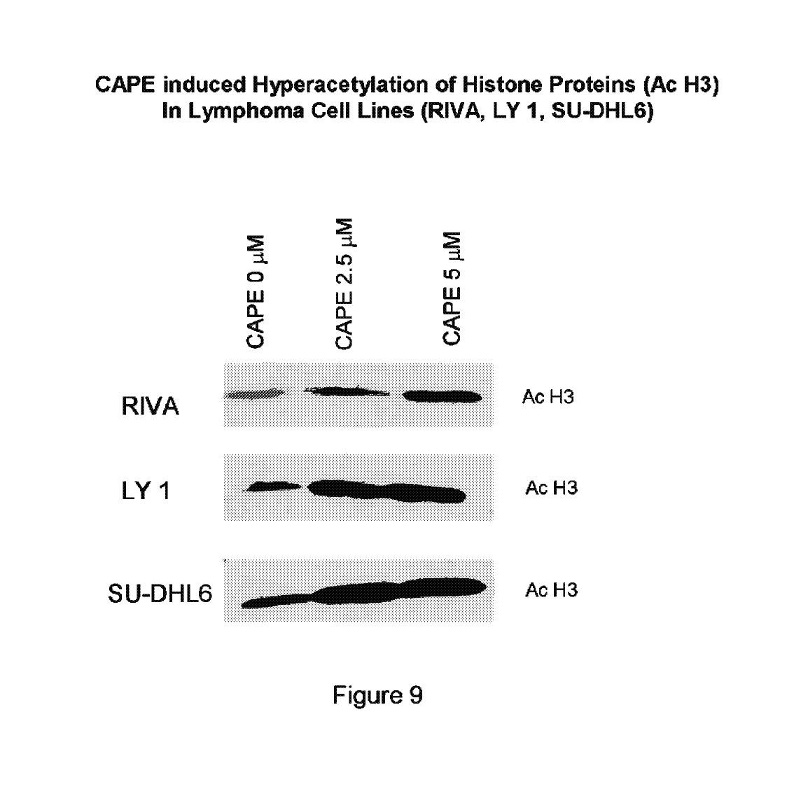

As shown in FIG. 2, CAPE and Propolis are HDAC inhibitors in the cell lines tested including MDA-231 (ER−/PR−/Her2−, triple negative breast cancer) and MCF-7 (ER+/PR). These cell lines serve as in vitro breast cancer models. These data are from in vitro experiments, using Western blotting techniques that clearly demonstrate the accumulation of acetylated histone proteins. This typically signifies the inhibition of histone deacetylases and is used as a readout of this process via Western blotting. Additionally, we show that HDAC inhibition is recapitulated in the peripheral blood mononuclear cells of a healthy volunteer after oral ingestion of CAPE-containing propolis for three weeks (FIG. 2). Further, FIGS. 3 and 4 provide additional evidence of the epigenetic changes that occur with CAPE and propolis resulting from HDAC inhibition. FIG. 3 depicts the downregulation of the estrogen receptor (ER) and progesterone receptor (PR) in MCF-7 cells and the downregulation of the epidermal growth factor receptor (EGFR) in MDA-231 cells (FIG. 4B). The re-expression of a previously silenced gene, (ER), a key function of HDAC inhibition, is depicted by the upregulation of the estrogen receptor in the ER-negative MDA-231 cell line (FIG. 4A). Furthermore, we have shown the HDAC inhibitor activity is not just restricted to breast cancer, but also in lymphoma (FIG. 9).

Collectively, these results demonstrate that CAPE and propolis are HDAC inhibitors, and exhibit activity in the range of that shown and accepted for another established inhibitor of histone deacetylases (LBH 589; see FIG. 4). Further, this epigenetic mechanism of action is not limited to cancer, but has repercussions in a wide variety of clinical scenarios, including multiple other cancers like prostate cancer, head and neck cancers, melanoma, lung cancers and leukemia. As described herein, CAPE and Propolis can be used in these scenarios in combination with drugs if the patient has become refractory. These drugs include the class of anti-androgens for prostate cancer; erlotinib (Tarceva) for lung cancer; or in combination with chemotherapy in melanoma, head and neck cancers and leukemia. In addition, some viral infections are sensitive to HDAC inhibitors. Additional information pertaining to the above is known in the art, see, for example, Demestre et al. (2009) Phytother Res 23:226-30; Wardley et al. (2010) J Clin Oncol 28:15s, Supp. Abstract 1052; and Qian et al. (2007) Prostate 67:1182-93, the content of each of which is incorporated herein by reference in its entirety.

As shown in FIG. 2, the intact natural product propolis is a better HDAC inhibitor than the single agent CAPE. This has been observed with select other naturopathic formulations. Interestingly, the aliphatic backbone structure of CAPE is similar to that of chemicals known to possess this unusual activity (i.e., valproic acid, butyric acid, etc). The enhancement of activity may be due to the additional caffeic acid analogs, or the hydrolyzed products from CAPE metabolism in the naturopathic formulation, leading to enhanced HDAC inhibitor effects, as well as better solubility in the vehicle used. Finally, it could be that CAPE is more stable as a molecular compound when it is present in its natural product form and, therefore, more potent. In sum, FIGS. 1, 6-8 reveal a more potent propolis cytotoxic activity in breast cancer and lymphoma cells as compared with that of CAPE.

The knowledge that Propolis/CAPE is an HDAC inhibitor suggests the use of therapeutic combination strategies. The HDAC inhibitor class of drugs is well known to act synergistically or additively with standard chemotherapeutic/targeted therapies currently in use in oncology. Specifically in breast cancer, our pre-clinical evidence (see, e.g., FIGS. 3-5) of the epigenetic effects of CAPE and Propolis-induced decrease in hormone receptors, ER and PR, could potentially lead to anti-hormonal effects in hormonal therapy refractory cells used alone or in combination with chemotherapy. Moreover, the re-expression of ER receptor as induced by CAPE and propolis in triple negative breast cancer (TNBC) may render these patients susceptible to anti-estrogen therapy with Tamoxifen, if used in combination. Likewise, a decrease in over-expression of EGFR in TNBC and phosphorylated Her2/neu in Her2-overexpressing cells make it an excellent candidate as targeted therapy in combination with known EGFR inhibitors, like cetuximab (Erbitux), Panitumumab (Vectibix) and anti-Her2-signaling drugs, like trastuzumab (Herceptin) or Lapatinib. The use of HDAC inhibitor combination therapies is described in references listed below, which are incorporated herein by reference in their entireties.

### REFERENCES

1. HDAC inhibitor vorinostat enhances the antitumor effect of gefitinib in squamous cell carcinoma of head and neck by modulating ErbB receptor expression and reverting EMT. Bruzzese F, Leone A, Rocco M, Carbone C, Piro G, Caraglia M, Di Gennaro E, Budillon A. J Cell Physiol. 2010 Dec. 9.
2. Novel Chimeric Histone Deacetylase Inhibitors: A Series of Lapatinib Hybrides as Potent Inhibitors of Epidermal Growth Factor Receptor (EGFR), Human Epidermal Growth Factor Receptor 2 (HER2), and Histone Deacetylase Activity. Mahboobi S, Sellmer A, Winkler M, Eichhorn E, Pongratz H, Ciossek T, Baer T, Maier T, Beckers T. J Med Chem. 2010 Dec. 23; 53(24):8546-55. Epub 2010 Nov. 16.
3. A phase I study of the safety and pharmacokinetics of the histone deacetylase inhibitor belinostat administered in combination with carboplatin and/or paclitaxel in patients with solid tumours. Lassen U, Molife L R, Sorensen M, Engelholm S A, Vidal L, Sinha R, Penson R T, Buhl-Jensen P, Crowley E, Tjornelund J, Knoblauch P, de Bono J S. Br J Cancer. 2010 Jun. 29; 103(1):12-7. Epub 2010 Jun. 15.
4. Histone deacetylase inhibitor romidepsin enhances anti-tumor effect of erlotinib in non-small cell lung cancer (NSCLC) cell lines. Zhang W, Peyton M, Xie Y, Soh J, Minna J D, Gazdar A F, Frenkel E P. J Thorac Oncol. 2009 February; 4(2):161-6.
5. Phase II data for entinostat, a class 1 selective histone deacetylase inhibitor, in patients whose breast cancer is progressing on aromatase inhibitor therapy. Wardley et al. J Clin Oncol 28:15s, 2010 (suppl; abstract 1052).
6. Antitumor activity of the histone deacetylase inhibitor MS-275 in prostate cancer models. Qian et al. Prostate 2007; 67:1182-93.
7. Genistein cooperates with the histone deacetylase inhibitor vorinostat to induce cell death in prostate cancer cells. Phillip et al. BMC Cancer 2012; 12:145.
8. Valproic acid: Growth inhibition of head and neck cancer by induction of terminal differentiation and senescence. Gan et al. doi: 10.1002/hed.21734.
9. Clinical and translational studies of depsipeptide (romidepsin), a histone deacetylase (HDAC) inhibitor, in patients with squamous cell carcinoma of the head and neck (SCCHN): New York Cancer Consortium Trial P6335. Haigentz et al. J Clin Oncol 2007; ASCO Meeting Proceedings Part I. 25:No. 18S (June 20 Supp):6065.
10. Histone deacetylase inhibitors in lymphoma. Copeland et al. 2010. Curr Opin Oncol 22:431-6.
11. Jotte, R., S. Witta, et al. (2010). “Molecular analysis identifies a subset of non-small cell lung cancer patients with differential sensitivity to histone deacetylase inhibitor (hdaci)/epidermal growth factor receptor tyrosine kinase inhibitor (egfr-tki) treatment.” 2010 Chicago Multidisciplinary Symposium in Thoracic Oncology. LBOA3 presentation.
12. Gojo, I., A. Jiemjit, et al. (2007) “Phase 1 and pharmacologic study of MS-275, a histone deacetylase inhibitor, in adults with refractory and relapsed acute leukemias.” Blood. 109(7):2781-90.
13. Hauschild, A., U. Trefzer, et al. (2008). “Multicenter phase II trial of the histone deacetylase inhibitor pyridylmethyl-N-{4-[(2-aminophenyl)-carbamoyl]-benzyl}-carbamate in pretreated metastatic melanoma.” Melanoma Res. 18(4):274-78.
14. Tan et al. Novel histone deacetylase inhibitors in clinical trials as anti-cancer agents. Journal of Hematology & Oncology 2010, 3:5.
15. Wightman et al. HDAC inhibitors in HIV. Immunology and Cell Biology (2012) 90, 47-54; doi:10.1038/icb.2011.95; published online 15 Nov. 2011.
16. Archin et al. Expression of latent HIV induced by the potent HDAC inhibitor suberoylanilide hydroxamic acid. AIDS Res Hum Retroviruses. 2009 February; 25(2):207-12.

### Example II

Background:

TNBC is a high mortality disease with a paucity of therapeutics. Our preclinical data used CAPE, a main component of propolis, a honeybee product credited with antiinflammatory, antioxidant, and antitumor properties. CAPE, which is innocuous to normal human mammary epithelial cells, inhibits growth of MDA-MB-231 (MDA-231) cells, MDR gene expression, NF-kB, EGFR, and VEGF. We used MDA-231 cells, a model of human TNBC, to show that CAPE can combat TNBC as a therapeutic and/or adjuvant to chemotherapeutic drugs, like Taxol. Cancer stem cells (CSC) are implicated in tumor metastasis and recurrence, thus, agents impairing their self-renewal, could be invaluable as novel cancer therapeutics. We show that CAPE could be such an agent, because it inhibits CSC growth and self renewal. We propose that the mechanism for this inhibition is due to the induction of CSC apoptosis.

Methods:

Nude mice bearing MDA-231 xenografts were fed CAPE-containing diets and/or treated topically with CAPE, and tumors measured twice/week. bCSC (breast CSC) were isolated from MDA-231 cells and xenografts, and propagated as mammospheres. Cell growth was analyzed using MTT and cell cycle by flow cytometry. Apoptosis was determined by co-staining bCSC with Annexin V and propidium iodide.

Results:

Dietary CAPE significantly inhibited growth of MDA-231 xenografts. Subsequent topical CAPE treatment further decreased tumor volume. MDA-231 cells contain bCSC thought to be responsible for metastasis and recurrence. CAPE decreased bCSC clonal growth and inhibited their self-renewal and that of progenitors, as evidenced by decreased growth in soft agar after pretreatment with 20 μM CAPE, but apoptosis was decreased starting at a higher dose (40 μM).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for treating a patient with cancer, the method comprising administering to the patient a therapeutically effective amount of propolis or CAPE in combination with an agent used in hormonal therapy of cancer, wherein administration of the propolis or CAPE and the agent reduces the number of cancer cells or the tumor burden in the patient, thereby treating the patient.

2. The method of claim 1, wherein the cancer is breast cancer or prostate cancer.

3. The method of claim 2, wherein the breast cancer is a triple negative breast cancer (TNBC).

4. The method of claim 3, wherein the agent is an anti-estrogen therapeutic.

5. The method of claim 4, wherein the anti-estrogen therapeutic is Tamoxifen, Faslodex or an Aromatase Inhibitor.

6. The method of claim 4, wherein the propolis or CAPE restores sensitivity to the anti-estrogen therapeutic by inducing re-expression of estrogen receptors on the TNBC.

7. The method of claim 3, wherein the triple negative breast cancer (TNBC) over-expresses epidermal growth factor receptor (EGFR).

8. The method of claim 7, wherein the propolis or CAPE restores sensitivity to the EGFR inhibitor.

9. The method of claim 8, wherein the agent is an EGFR inhibitor.

10. The method of claim 9, wherein the EGFR inhibitor is cetuximab (Erbitux), panitumumab (Vectibix), and erlotinib (Tarceva).

11. The method of claim 1, wherein the cancer is refractory to at least one therapeutic regimen directed to eradicating the cancer.

12. The method of claim 2, wherein the breast cancer is refractory to hormonal therapy.

13. The method of claim 12, wherein the breast cancer is Her2+.

14. The method of claim 13, wherein the breast cancer is refractory to an anti-Her2 signaling drug and the agent is the anti-Her2 signaling drug and the propolis or CAPE restores sensitivity to the anti-Her2 signaling drug.

15. The method of claim 14, wherein the anti-Her2 signaling drug is trastuzumab (Herceptin) or Lapatinib, or Pertuzumab.

16. The method of claim 1, further comprising treating the patient with chemotherapy.

17. A composition comprising propolis or CAPE and an agent used in hormonal therapy of cancer and a pharmaceutically acceptable excipient.

18. A method for treating a patient with cancer, wherein the cancer is refractory to at least one therapeutic regimen directed to eradicating the cancer, the method comprising administering to the patient a therapeutically effective amount of propolis or CAPE in combination with an agent used in the at least one therapeutic regimen directed to eradicating the cancer, wherein administration of the propolis or CAPE and the agent reduces the number of cancer cells or the tumor burden in the patient, thereby treating the patient.

19. The method of claim 18, wherein the cancer is prostate cancer, head and neck cancer, melanoma, lung cancer or leukemia.

20. The method of claim 19, wherein the cancer is prostate cancer and the prostate cancer is refractory to anti-androgens (Lupron, Zoladex, Casodex, nilandrone, flutamide, abiraterone); the cancer is head and neck cancer and the head and neck cancer is refractory to Cetuximab or chemotherapy+/−radiation; the cancer is melanoma and the melanoma is refractory to chemotherapy or Ipilumumab or BRAF inhibitors such as vemurafenib; the cancer is lung cancer and the lung cancer is refractory to chemotherapy or Alimta or Erlotinib (Tarceva); or the cancer is leukemia and the leukemia is refractory to chemotherapy.

21-40. (canceled)
